(19) United States
(12) Patent Application Publication
(10) Pub. No.: US 2025/0239072 A1
(43) Pub. Date: Jul. 24, 2025

(54) THERMAL/VISIBLE IMAGER FOR CROP STRESS DETECTION

(71) Applicant: The United States of America, as represented by the Secretary of Agriculture, Washington, DC (US)

(72) Inventors: JOAQUIN J. CASANOVA, PULLMAN, WA (US); SUSAN A. O'SHAUGHNESSY, AMARILLO, TX (US); PAUL D. COLAIZZI, AMARILLO, TX (US); COLLIN S. CAMPBELL, PULLMAN, WA (US)

(21) Appl. No.: 18/417,234

(22) Filed: Jan. 19, 2024

(51) Int. Cl. G06V 20/10; G01N 21/84; G01N 33/00; G06T 7/33; G06V 10/26; G06V 10/54; G06V 10/56; G06V 10/82

(52) U.S. Cl. CPC G06V 20/188; G01N 21/84; G01N 33/0098; G06T 7/337; G06V 10/26; G06V 10/54; G06V 10/56; G06V 10/82; G01N 2021/8466; G06T 2207/10024; G06T 2207/10048; G06T 2207/20084; G06T 2207/30188

(57) ABSTRACT

Method, apparatus, and computer program product are disclosed for estimating evapotranspiration (ET) using thermal and optical images. In some embodiments, a color image and a thermal image are acquired substantially simultaneously, each capturing a target of interest associated with an agricultural crop. Features, including color features and/or texture features, are extracted from the color image. The color image is segmented into surface temperature components based on the extracted features. The surface temperature components are selected from the group consisting of sunlit soil, sunlit residue, sunlit vegetation, sunlit snow, shaded soil, shaded residue, shaded vegetation, and shaded snow. The color image and the thermal image are co-registered to provide a registered thermal image. A component temperature is assigned to each of the surface temperature components by applying component masks to the registered thermal image. ET is estimated based on the component temperatures using an energy balance model or other ET model.

### BACKGROUND OF THE INVENTION

Disclosed are method, apparatus, and computer program product for estimating evapotranspiration (ET) using thermal images and optical images. In some embodiments of the disclosure, a color image and a thermal image are acquired substantially simultaneously, each capturing a target of interest associated with an agricultural crop. Features, including color features and/or texture features, are extracted from the color image. The color image is segmented into surface temperature components based on the extracted features. The surface temperature components are selected from the group consisting of sunlit soil, sunlit residue, sunlit vegetation, sunlit snow, shaded soil, shaded residue, shaded vegetation, and shaded snow. The color image and the thermal image are co-registered to provide a registered thermal image. A component temperature is assigned to each of the surface temperature components by applying component masks to the registered thermal image. ET is estimated based on the component temperatures using an energy balance model or other ET model.

Understanding heat and water stress on field crops is an increasingly important consideration in management decisions. In dryland cropping systems, knowledge of heat stress can help detect drought tolerant cultivars and disambiguate other sources of stress, such as nutrient deficiencies, as well as allow growers to better assess planting and harvest dates to maximize use of stored water. Meanwhile, in irrigated crops, precision irrigation through stress monitoring has proven an effective approach to save water resources. For example, detection of crop water stress can be used to regulate irrigation scheduling in dry regions. In either case, surface temperature measurements can act as an indicator of crop water stress, however, current crop water stress monitoring and evapotranspiration (ET) estimation typically relies on approximations based on single radiometer measurements rather than imagery, but methods like infrared thermometry give a mix of crop and soil temperatures that may be sunlit or shaded.

Although the same input data are commonly used for energy balance models (e.g., TSEB) and stress indices (e.g., CWSI), they output different variables that have different physical meanings. The TSEB outputs evaporation and transpiration, and the CWSI outputs an index that theoretically should range from zero (no water stress) to one (complete water stress, no water available for ET).

Combined thermal and visible imagery allows for more accurate ET assessment in early growth stages, as well as identification of crop biotic stressors (e.g., disease or weeds). Such imagers exist but are typically expensive and, with rare exception, are not adapted for agricultural applications. Two such exceptions are Osroosh et al. (2018) and Drew et al. (2019). Osroosh et al. developed a low-cost thermal-RGB imager for use in agricultural crop monitoring applications. In Osroosh et al., the image-processing algorithm outputs the average temperature of sunlit leaves and percent canopy coverage. Drew et al. developed a multi-band sensor for crop temperature measurement that combines a miniature long wavelength infrared (LWIR) camera with a visible, or red-green-blue (RGB), camera. In Drew et al., a processing algorithm outputs a temperature measurement more representative of the crop canopy by removing shaded areas and soil from thermal images. Applicants are unaware of any combined thermal and visible imagers that include the necessary image processing algorithms for crop water stress monitoring and ET estimation.

### SUMMARY OF THE INVENTION

Disclosed are method, apparatus, and computer program product for estimating evapotranspiration (ET) using thermal images and optical images. In some embodiments of the disclosure, a color image and a thermal image are acquired substantially simultaneously, each capturing a target of interest associated with an agricultural crop. Features, including color features and/or texture features, are extracted from the color image. The color image is segmented into surface temperature components based on the extracted features. The surface temperature components are selected from the group consisting of sunlit soil, sunlit residue, sunlit vegetation, sunlit snow, shaded soil, shaded residue, shaded vegetation, and shaded snow. The color image and the thermal image are co-registered to provide a registered thermal image. A component temperature is assigned to each of the surface temperature components by applying component masks to the registered thermal image. ET is estimated based on the component temperatures using an energy balance model or other ET model.

In accordance with some embodiments of the disclosure, a dual camera device includes a miniature thermal camera and a miniature red-green-blue (RGB) camera integrated with a single board computer (SBC). Firmware is written so that the camera acquires an image using the RGB camera, immediately and automatically followed by an image from the thermal camera to capture the same target of interest. Firmware co-registers the two images and estimates fractions of plant canopy cover, residue, and bare soil components, both sunlight and shadowed, and the component temperatures. From this information, estimates of ET using an energy balance model, such as a two-source energy balance (TSEB) model or an extension of TSEB model that is extended to include at least one additional source, or other ET model.

In some embodiments, an inexpensive color/thermal imager is disclosed that extracts surface specific temperatures using image segmentation. The device was tested at two sites in different cropping systems (Pullman, Washington and Bushland, Texas) and measured the temperatures of soil, residue, vegetation, and snow, under both shadowed and sunlit conditions. A multilayer perceptron was optimized and trained to segment images, using image features, including three different color spaces, and three different texture features, all applied at three different scales (i.e., the image features at different spatial scales). The trained model had accuracies and F1 scores of 0.8922 and 0.8923 for an initial prototype with lower resolution images, and 0.8840 and 0.8846 for a higher-resolution version. Segmented components had surface temperatures that indicate shadow was cooler than sunlit portions, vegetation was cooler than soil, and residue was hotter than soil, all as expected.

This summary is provided to introduce a selection of concepts in a simplified form that are further described below in the detailed description. This summary is not intended to identify key features of the claimed subject matter, nor is intended as an aid in determining the scope of the claimed invention.

### BRIEF DESCRIPTION OF THE SEVERAL VIEWS OF THE DRAWINGS

Embodiments will hereinafter be described in conjunction with the appended drawings, where like designations denote like elements.

FIG. 1 depicts an exemplary system for estimating evapotranspiration (ET) using a dual smart camera (DSC), according to one or more embodiments.

FIG. 2 is a flow diagram of an illustrative method of estimating evapotranspiration (ET) using a color image and a thermal image, according to one or more embodiments.

FIG. 3 is a flow diagram of an illustrative method of training and testing a multilayer perceptron (MLP) model used for segmenting a color image into surface temperature components based on features extracted from the color image, according to one or more embodiments.

FIG. 4 is a block diagram illustrating a representation of an exemplary computer system for performing a computer-implemented method of estimating evapotranspiration (ET) using a color image and a thermal image, according to one or more embodiment.

FIG. 5 is a conceptual diagram illustrating a two-source energy balance (TSEB) model with series resistances for a row crop, which TSEB model may be employed in conjunction with a dual smart camera (DSC) to estimate evapotranspiration (ET), according to one or more embodiments.

FIG. 6 is a conceptual diagram illustrating an extension of a two-source energy balance (TSEB) model (i.e., the TSEB model is extended to include at least one additional source, such as crop residue) with series resistances for a row crop, which extended TSEB model may be employed in conjunction with a dual smart camera (DSC) to estimate evapotranspiration (ET), according to one or more embodiments.

### DETAILED DESCRIPTION

Disclosed are method, apparatus, and computer program product for estimating evapotranspiration (ET) using thermal images and optical images. In some embodiments of the disclosure, a color image and a thermal image are acquired substantially simultaneously, each capturing a target of interest associated with an agricultural crop. Features, including color features and/or texture features, are extracted from the color image. The color image is segmented into surface temperature components based on the extracted features. The surface temperature components are selected from the group consisting of sunlit soil, sunlit residue, sunlit vegetation, sunlit snow, shaded soil, shaded residue, shaded vegetation, and shaded snow. The color image and the thermal image are co-registered to provide a registered thermal image. A component temperature is assigned to each of the surface temperature components by applying component masks to the registered thermal image. ET is estimated based on the component temperatures using an energy balance model or other ET model.

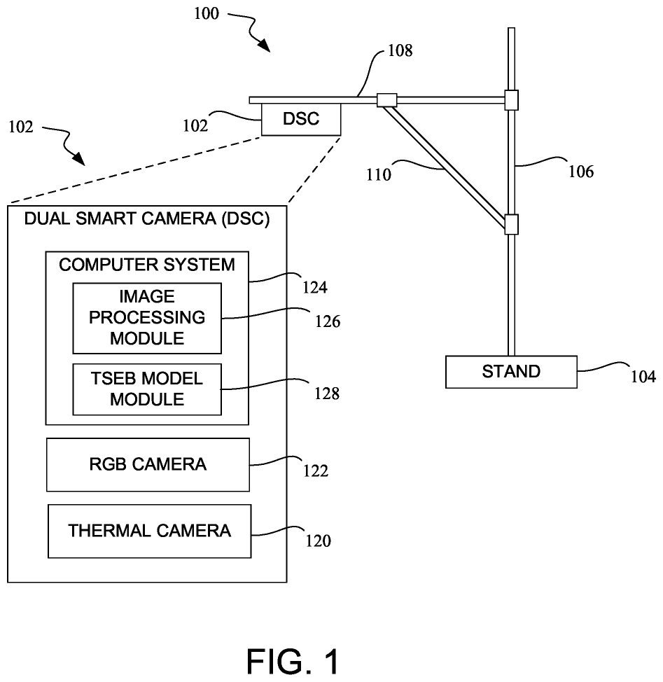

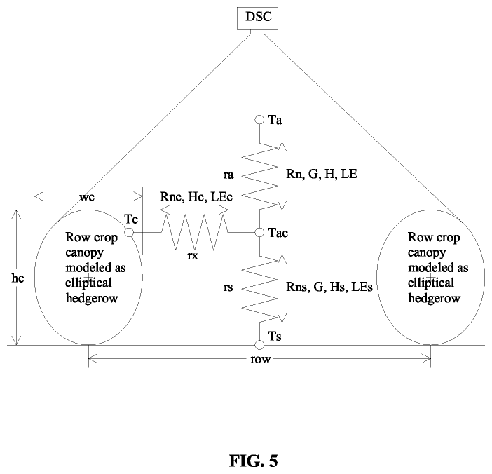

FIG. 1 depicts an exemplary system 100 for estimating evapotranspiration (ET) using a dual smart camera (DSC) 102, according to one or more embodiments. The DSC 102 may be supported above a target of interest associated with an agricultural crop, such as a row crop. For example, as best seen in FIG. 5, the DSC 102 may be supported between two row crop canopies at a predetermined distance (e.g., 1.5-2.5 m) above the row crop canopies. In the exemplary system 100 depicted in FIG. 1, the DSC 102 is supported by a stand the includes a weighted base 104, a post 106 (e.g., aluminum pole) vertically projecting from the weighted base 104, a beam 108 (e.g., aluminum pole) extending horizontally from the beam 108, and a brace 110 (e.g., aluminum) extending between the post 106 and the beam 108. The stand illustrated in FIG. 1 is exemplary. One skilled in the art will appreciate that other support mechanisms may be used to support the DSC 102 in lieu of the exemplary stand illustrated in FIG. 1. For example, the weighted base 108 of the stand illustrated in FIG. 1 may be replaced by a weighted tripod arrangement.

In the embodiment depicted in FIG. 1, the DSC 102 includes a thermal camera 120, a red-green-blue (RGB) camera 122, and a computer system 124 or other computing device, packaged together within a single housing. The DSC 102 is mounted on the support beam 108 with the field of view of the DSC 102 pointed downward capturing images of the target of interest. The field of view of the DSC 102, as best seen in FIG. 5, includes both the field of view of the thermal camera 120 and the field of view of the RGB camera 122. Preferably, the field of view of the thermal camera 120 is substantially identical to the field of view of the RGB camera 122. In some embodiments, the housing of the DSC 102 may include one or more ports through with the thermal camera 120 and the RGB camera 122 capture images of the target of interest.

The thermal camera 120 and the RGB camera 122 are conventional and commercially available.

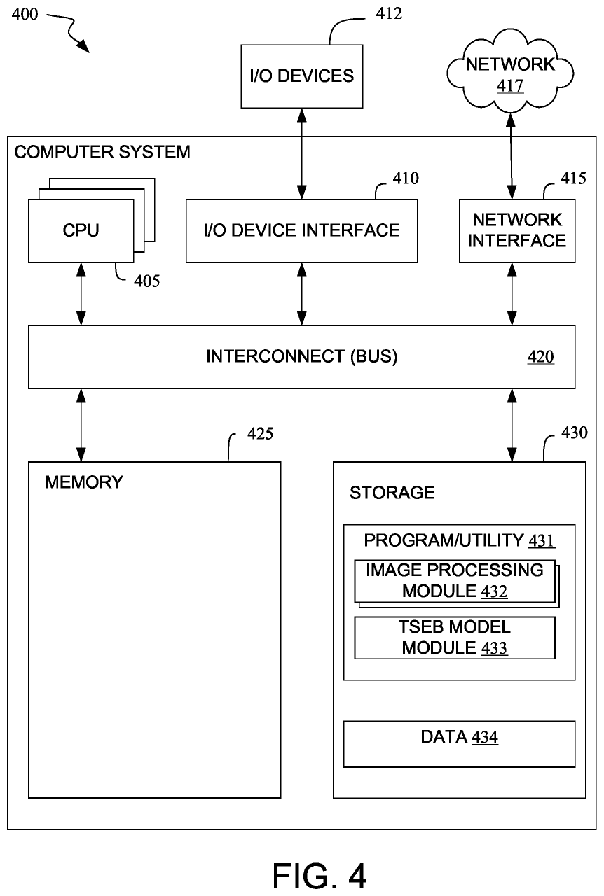

A computing device, such as a computer system 124, is operatively connected to the thermal camera 120 and the red-green-blue (RGB) camera 122. The computer system 124 is conventional and commercially available. For example, the computer system 124 may be a single-board computer (SBC). A representation of a suitable example of such a computer system is depicted in FIG. 4.

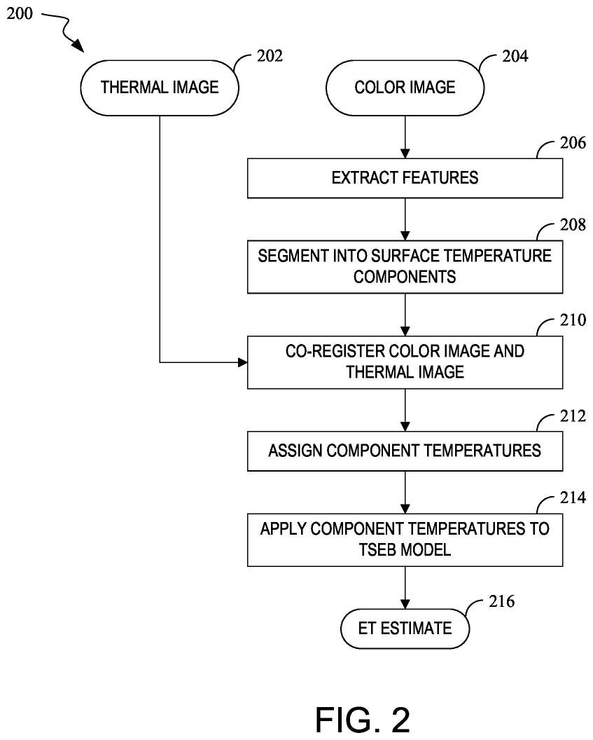

FIG. 2 is a flow diagram of an illustrative method 200 of estimating evapotranspiration (ET) using a thermal image 202 and a color image 204, according to one or more embodiments. The method 200 sets forth the preferred order of the blocks. It must be understood, however, that the various blocks may occur at any time relative to one another.

The method 200 begins by acquiring the color image 204 that captures a target of interest associated with an agricultural crop and acquiring the thermal image 202 that captures the target of interest at substantially the same time as the color image 204. For example, firmware may be written so that the DSC acquires the color image 204 using the RGB camera, immediately and automatically followed by the thermal image 202 from the thermal camera to capture the same target of interest. In some embodiments, the color image 204 and the thermal image 202 are acquired every 0.5-1 h to provide a color image time series and a thermal image time series capturing the target of interest over time. More generally, a frequency of image capture within the range of about 0.5 h to about 2 h is suitable, but a frequency of image capture within the range of about 0.5 h to about 1 h is preferred.

The method 200 continues by extracting one or more features from the color image 204 (block 206). In some embodiments, extracting one or more features from the color image 204 may include extracting color features and/or texture features. Color features may include, for example, three different color spaces: RGB (red, green, blue); HSV (hue, saturation, value); and LaB (L*a*b* colorspace components)—for a total of nine bands (i.e., red, green, blue, hue, saturation, value, and L*a*b* colorspace components). Texture features may include, for example, three different texture features: standard deviation; linear binary pattern (LBP); and Laplacian at three scales. For example, extracting one or more features may include, extracting, for each pixel, features including an individual pixel value, Gaussian blur at two scales, standard deviation at three scales, linear binary pattern at three scales, and Laplacian at three scales in each of nine bands, wherein the nine bands include red, green, blue, hue, saturation, value, and L*a*b* colorspace components.

The CIELAB color space, also referred to as L*a*b*, is a color space defined by the International Commission on Illumination (abbreviated CIE) in 1976. The L*a*b* color space expresses color as three values: L* for perceptual lightness, as well as a* and b* axes for the four unique colors of human vision: red, green, blue, and yellow, where red and green form an opponent pair and blue and yellow form an opponent pair. The perceptual lightness value, L*, defines black at 0 and white at 100. The a* axis is relative to the green-magenta opponent colors, with negative values toward green and positive values toward magenta. The b* axis represents the blue-yellow opponents, with negative numbers toward blue and positive toward yellow.

The method 200 continues by segmenting the color image 204 into surface temperature components based on the one or more features extracted from the color image 204 (block 208). For example, the surface temperature components may be selected from the group consisting of sunlit soil, sunlit residue, sunlit vegetation, sunlit snow, shaded soil, shaded residue, shaded vegetation, and shaded snow. In some embodiments, segmenting the color image 204 includes performing classification by inputting a reduced feature set into a multi-layer perceptron.

The method 200 continues by co-registering the color image 204 and the thermal image 202 to provide a registered thermal image (block 210). In some embodiments, co-registering the color image 204 and the thermal image 202 includes using a thermally reflective object (e.g., a foil covered square) that appears in the color image 204 and the thermal image 202, matching a plurality of features of the thermally reflective object between the color image 204 and the thermal image 202, and using the plurality of features of the thermally reflective object to calculate an affine transform to warp the thermal image 202 to match the color image 204.

The method 200 continues by assigning component temperatures by applying component masks to the registered thermal image to assign a component temperature to each of the surface temperature components (block 212). In some embodiments, assigning component temperatures includes constructing a time series TS-Component for each of the surface temperature components. For example, a time series TS-Component may be constructed for the surface temperature of the sunlit soil, the sunlit residue, the sunlit vegetation, the sunlit snow, the shaded soil, the shaded residue, the shaded vegetation, and the shaded snow.

The method 200 continues by applying the component temperatures to an energy balance model or other ET model (block 214) to provide an ET estimate 216. In some embodiments, applying the component temperatures to an energy balance model in block 214 may include employing a TSEB model. A representation of a suitable example of such a TSEB is depicted in FIG. 5.

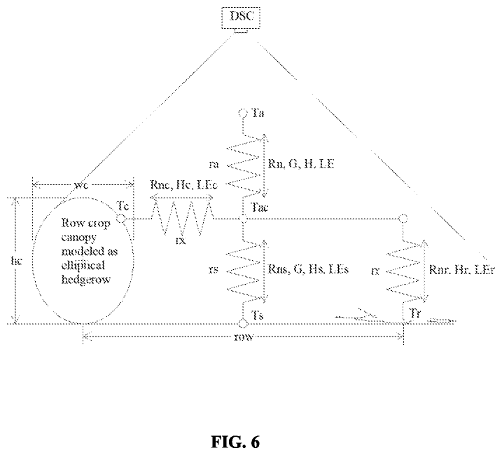

In some embodiments, applying the component temperatures to an energy balance model in block 214 may include employing an extension of a TSEB model extended to include at least one additional energy source. A representation of a suitable example of such an extended TSEB model is depicted in FIG. 6.

In accordance with some embodiment of the disclosure, building, training, and testing the energy balance model may be accomplished by, for example, fitting model parameters, including displacement lengths and canopy, residue, soil, and snow albedos and emissivity, using data from individual crops representative of the agricultural crop, in comparison with independently measured ET.

In some embodiments, applying the component temperatures to the other ET model in block 214 includes utilizing a recurrent neural network (RNN) or multilayer perceptron (MLP) that estimates ET based on the component temperatures and local meteorological time series. See, for example, the discussion of a RNN in Sherstinsky, Physica D: Nonlinear Phenomena, 404, 132306 (2020).

In accordance with some embodiments of the disclosure, building, training, and testing the other ET model may be accomplished by, for example, fitting size and structure, as well as weights, of a recurrent neural network (RNN) or multilayer perceptron (MLP) using data from individual crops representative of the agricultural crop, in comparison with independently measured ET.

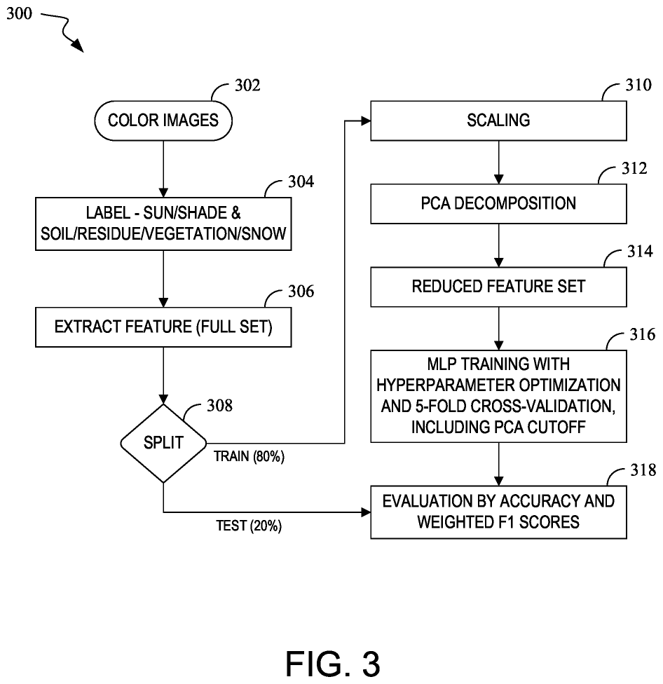

The model used for image segmentation in block 208 in the illustrative method 200 depicted in FIG. 2 is a multi-layer perceptron (MLP) model, which is a form of neural network. An illustrative method 300 of training and testing the MLP model is depicted in FIG. 3.

FIG. 3 is a flow diagram of an illustrative method 300 of training and testing a multi-layer perceptron (MLP) model used for segmenting a color image into surface temperature components based on features extracted from the color image, according to one or more embodiments. The method 300 sets forth the preferred order of the blocks. It must be understood, however, that the various blocks may occur at any time relative to one another.

The method 300 begins by acquiring the color images 302 that captures a target of interest associated with an agricultural crop. The color images 302 serve as the basis for obtaining data for training and testing the MLP model. For example, firmware may be written so that the DSC acquires the color images 302 to capture the same target of interest using the RGB camera. In some embodiments, a color image 302 is acquired every 0.5-1 h to provide a color image time series capturing the target of interest over time. More generally, a frequency of image capture within the range of about 0.5-2 h is suitable, but a frequency of image capture within the range of about 0.5-1 hr is preferred.

The method 300 continues by labeling the color images 302 (block 304). In some embodiments, color images may be manually labeled to serve as training data for a machine vision algorithm for segmentation. For example, a color image may be created with soil, residue, vegetation, and snow labels, then a separate image may be created with sunlit and shadow labels. The two labeled images may be combined to create an image with eight possible classes. In some embodiments, images may be selected throughout the season to ensure a good diversity of vegetation cover and lighting conditions.

The method 300 continues by extracting features from the color images 302 (block 306). For example, an initial set of features (individual pixel values, Gaussian blur at two scales, standard deviation at three scales, linear binary pattern at three scales, and Laplacian at three scales) may be calculated for each image in the training data in each of nine bands (red, green, blue, hue, saturation, value, and L*a*b* colorspace components). OpenCV, for example, may be used to calculate features and Scikit-learn may be used for the segmentation model training and testing.

The method 300 continues by splitting the data obtained in blocks 304 and 306 for purposes of training (80%) and for purposes of testing (20%) (block 308). In other words, the 108 features ((9 bands×3 textures×3 scales)+(9 bands×1 pixel value)+(9 bands×blur at 2 scales)) for each pixel obtained in block 106, and corresponding pixel labels obtained in block 108 are split into 80% for training and 20% for testing. These percentages are exemplary. One skilled in the art will appreciate that other percentages may be utilized in lieu of an 80%/20% split.

The method 300 continues by scaling the training data set (block 310).

The method 300 continue by performing principal component analysis (PCA) decomposition of the feature set into components (block 312).

The method 300 continues by applying PCA to reduce the feature set, with the components accounting for a certain percentage of the variance retained (block 314).

The method 300 continues by multilayer perceptron (MLP) training with hyperparameter optimization and 5-fold cross-validation, including PCA cutoff (block 316). For example, the reduced feature set may be fed into a MLP with one or more hidden layers. The hyperparameters of the MLP (nodes per layer and activation function) and the percentage used in the PCA cutoff may be optimized using a grid search.

Finally, the method 300 ends with the optimized model MLP being evaluated with the test set, using test statistics of weighted F1 and accuracy scores (block 318).

FIG. 4 is a block diagram illustrating an exemplary representation of a computer system 400 for performing a computer-implemented method of estimating evapotranspiration (ET) using a color image and a thermal image. As shown, the computer system 400 includes, without limitation, at least one CPU 405, a network interface 415, an interconnect 420, a memory 425, and storage 430. The computer system 400 may also include an I/O device interface 410 used to connect I/O devices 412 (e.g., keyboard, display, mouse devices, thermal camera, such as thermal camera 120 in FIG. 1, and red-green-blue (RGB) camera, such as RGB camera 122 in FIG. 1) to the computer system 400.

Each CPU 405 retrieves and executes programming instructions stored in the memory 425 and storage 430. Similarly, the CPU 405 stores and retrieves application data residing in the memory 425 and storage 430. The network interface 415 is configured to transmit data via the communications network 417. The interconnect 420 is used to transmit programming instructions and application data between each CPU 405, I/O device interface 410, network interface 415, memory 425, and storage 430. The interconnect 420 may be one or more busses. CPU 405 is included to be representative of a single CPU, multiple CPUs, a single CPU having multiple processing cores, and the like. Memory 425 is generally included to be representative of a random access memory, e.g., SRAM, DRAM or Flash. Storage 430, such as a hard disk drive, solid state disk (SSD), or flash memory storage drive, may store non-volatile data. Although shown as a single unit, the storage 430 may be a combination of fixed and/or removable storage devices, such as fixed disc drives, removable memory cards, optical storage, SSD or flash memory devices, network attached storage (NAS), connections to storage area-network (SAN) devices, or to the cloud.

A program/utility 431, having a set (at least one) of program modules 432, may be stored in storage 430 by way of example, and not limitation, as well as an operating system, one or more application programs, other program modules, and program data. Each of the operating system, one or more application programs, other program modules, and program data or some combination thereof, may include an implementation of a networking environment. The program modules, such as image processing module 432 and TSEB model module 433, generally carry out the functions and/or methodologies of one or more embodiments as described herein. Storage 430 may also contain data 434, such as thermal image data and RGB image data.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In one or more embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to one or more embodiments. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

One or more embodiments may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g., an amount of storage space used by a user or a number of virtualized systems instantiated by the user). A user can access any of the resources that reside in the cloud at any time, and from anywhere across the Internet. In context of the present invention, a user may access applications or related data available in the cloud. For example, the nodes used to create a stream computing application may be virtual machines hosted by a cloud service provider. Doing so allows a user to access this information from any computing system attached to a network connected to the cloud (e.g., the Internet).

The energy balance of the substrate-canopy-atmosphere is typically based on equating available energy (commonly described as net radiation and surface soil heat flux) with sensible and latent heat fluxes, where stored energy and photosynthesis are assumed negligible:

$${R}_{N} - {G}_{0} = H + LE \qquad ( 1 )$$

where R_{N} is net radiation, G₀ is surface soil heat flux, H is sensible heat flux, and LE is latent heat flux (all units are W m⁻²). Using this sign convention, R_{N} is positive toward, and all other terms are positive away from, the canopy or substrate.

The Two-Source Energy Balance Model (TSEB) model was originally described by Norman et al. (1995) and Kustas et al. (1999) and has undergone further development by Colaizzi et al. (2012a, 2016a, and 2017). The TSEB model partitions each term into its canopy and substrate components, except for G₀, which only applies to the substrate (FIG. 5). Writing equation 1 separately for the canopy and substrate energy balances gives:

$${LE}_{C} = {R}_{N , C} - {H}_{C} \qquad ( 2 a )$$

$${LE}_{S} = {R}_{N , S} - {G}_{0} - {H}_{S} \qquad ( 2 b )$$

where the subscripts C and S refer to the canopy and substrate, respectively. As with most energy balance models, the TSEB model calculates the LE components as residuals of the other terms, and H components are calculated based on temperature gradients and resistances.

FIG. 5 is a conceptual diagram illustrating a two-source energy balance (TSEB) model with series resistances for a row crop, which TSEB model may be employed in conjunction with a dual smart camera (DSC) to estimate evapotranspiration (ET), according to one or more embodiments.

In FIG. 5, a DSC aimed obliquely has an elliptical footprint, which can be partitioned between soil and vegetation mathematically given that the vegetation is described as an elliptical hedgerow: w_{C}=width of crop row, h_{C}=height of crop row, T_{C}=temperature of canopy, row=orthogonal distance between rows, T_{S}=substrate surface temperature, T_{AC}=temperature in the canopy air space (approximate mean of canopy and air temperatures), and T_{A}=air temperature. The energy flux terms (R_{N}, G, H, and LE) are defined in equations 1, 2a, and 2b. Shadow/sunlit components are incorporated by accounting for their contributions in the thermal radiation balance.

Calculation procedures for R_{N,C} and R_{N,S} are given by Colaizzi et al. (2012a, 2012b). Briefly, these procedures use a combination of a radiative transfer model (Campbell and Norman, 1998) to calculate the shortwave radiation balance of the canopy and soil, and geometric models to account for the spatial distribution of row crop vegetation (Colaizzi et al., 2010).

A new method to calculate G₀ was developed by Colaizzi et al. (2016b). This method was used in the present embodiment as:

$${G}_{0} = \frac{{R}_{N , C} - {R}_{N , S , min}}{{R}_{N , C} - {R}_{N , S , min} \times ( {aR}_{N , S , max} - {R}_{N , S , min} ) + {R}_{N , S , min}} \qquad ( 3 )$$

where R_{N,S,min} and R_{N,S,max} are the minimum and maximum net soil radiation for a 24 h period (W m⁻²), respectively, and a is an empirical constant (dimensionless and found to be 0.31 for a clay loam soil at Bushland, Texas, by Colaizzi et al., 2016b, using a different data set). The rationale for this method was to account for sunlit and shaded soil in the crop interrow, which may comprise substantial positional variation of energy balance terms (e.g., Agam et al., 2012; Kool et al., 2014).

The sensible heat flux terms (H_{C} and H_{S}) were calculated as:

$$H = ρ {C}_{P} \frac{{T}_{AC} - {T}_{A}}{{r}_{A}} \qquad ( 4 a )$$

$${H}_{C} = ρ {C}_{P} \frac{{T}_{C} - {T}_{AC}}{{r}_{X}} \qquad ( 4 b )$$

$${H}_{S} = ρ {C}_{P} \frac{{T}_{S} - {T}_{AC}}{{r}_{S}} \qquad ( 4 c )$$

where ρ is the density of moist air (kg m⁻³), C_{P} is the specific heat of air (assumed constant at 1013 J kg⁻¹ K⁻¹), T_{C}, T_{A}, T_{S}, and T_{AC} are the temperatures of the canopy, air, soil, and air temperature within the canopy boundary layer, respectively (all in K), r_{A} is the aerodynamic resistance between the canopy and the air above the canopy (s m⁻¹), r_{X} is the resistance between the canopy and canopy boundary layer (s m⁻¹), and r_{S} is the resistance in the boundary layer immediately above the soil surface (s m⁻¹).

The T_{C} and T_{S} terms in equation 4b and 4c, respectively, are extracted from the DSC image. Since H=H_{C}+H_{S}, equation 4 results in:

$${T}_{AC} = \frac{\frac{{T}_{A}}{{r}_{A}} + \frac{{T}_{S}}{{r}_{S}} + \frac{{T}_{C}}{{r}_{X}}}{\frac{1}{{r}_{A}} + \frac{1}{{r}_{S}} + \frac{1}{{r}_{X}}} \qquad ( 5 )$$

Calculated LE_{S}, LE_{C}, and LE (W m⁻²) were converted to E, T, and ET (mm). For example, considering ET and 15-minute time steps:

$$ET = LE \frac{1000 \times 900}{( 1 {0}^{6} {ρ}_{W} λ )} \qquad ( 6 )$$

where 1000 converts m to mm, 900 converts time intervals of 1.0 s to 15 min, 10⁶ converts MJ to J, ρ_{W} is the density of water at 20° C. (1000 kg m⁻³), and λ is the latent heat of vaporization (MJ kg⁻¹), where λ=2.501-0.002361 T_{A}, and λ=˜2.44 MJ kg⁻¹.

FIG. 6 is a conceptual diagram illustrating an extension of a two-source energy balance (TSEB) model (i.e., the TSEB model is extended to include at least one additional source) with series resistances for a row crop, which extended TSEB model may be employed in conjunction with a dual smart camera (DSC) to estimate evapotranspiration (ET), according to one or more embodiments.

In FIG. 6, the subscript r indicates residue or mulch. For example, Tᵣ=temperature of residue. Snow can also be included similarly if present. In other respects, the extended TSEB depicted in FIG. 6 is identical to the TSEB depicted in FIG. 5, but with the addition of a parallel resistance between the canopy boundary layer and the substrate. That is, a DSC aimed obliquely has an elliptical footprint, which can be partitioned between soil and vegetation mathematically given that the vegetation is described as an elliptical hedgerow: w_{C}=width of crop row, h_{C}=height of crop row, T_{C}=temperature of canopy, row=orthogonal distance between rows, T_{S}=substrate surface temperature, T_{AC}=temperature in the canopy air space (approximate mean of canopy and air temperatures), and T_{A}=air temperature. The energy flux terms (R_{N}, G, H, and LE) are defined in equations 1, 2a, and 2b. Shadow/sunlit components are incorporated by accounting for their contributions in the thermal radiation balance.

The following examples are intended only to further illustrate the invention and are not intended to limit the scope of the invention as defined by the claims.

### EXAMPLES

In the following examples, a design for an inexpensive color/thermal imager is presented which extracts surface specific temperatures using image segmentation. The device was tested at two sites in different cropping systems (i.e., Pullman, Washington and Bushland, Texas) and measured the temperatures of soil, residue, vegetation, and snow, under both shadowed and sunlit conditions. A multilayer perceptron was optimized and trained to segment images, using image features, including three different color spaces, and three different texture features, all applied at three different scales (i.e., the image features at different spatial scales). The trained model had accuracies and F1 scores of 0.8922 and 0.8923, respectively, for an initial prototype with lower-resolution images, and 0.8840 and 0.8846, respectively, for a higher-resolution version. Segmented components had surface temperatures that indicate shadow was cooler than sunlit portions, vegetation was cooler than soil, and residue was hotter than soil, all as expected.

The use of trade, firm, or corporation names in this article is for the information and convenience of the reader. Such use does not constitute an official endorsement or approval by the USDA of any product or service to the exclusion of others that may be suitable.

### Materials and Methods

### Site Descriptions

Two test sites were used to test the DSC, one in east Washington and the other in the Panhandle of Texas. At both sites the DSC was internet connected using an Airlink RV50 cellular modem (Sierra Wireless, 2023).

### R. J. Cook Agronomy Farm, Pullman, Washington

The initial site of the DSC deployment was the Washington site at the R. J. Cook Agronomy Farm (CAF), located north of Pullman, WA (46° 47′N, 117° 5′W, 770 m above mean sea level). The CAF is a 37 ha site operated as part of the USDA Long-Term Agroecosystem Research (LTAR) network (Kleinman et al., 2018), with a comprehensive meteorological, harvest, and soil dataset (Huggins, 2015). The soil types are mostly Thatuna (a fine-silty, mixed, superactive, mesic Oxyaquic Argixeroll), Palouse (a fine-silty, mixed, superactive, mesic Pachic Ultic Haploxeroll), and Naff (a fine-silty, mixed, superactive, mesic Typic Argixeroll) (NRCS Web Soil Survey, 2023). The region has a Mediterranean climate with mean annual temperature of 9° C., rainfall of 518 mm, and snow of 831 mm (NOAA Climate Data Online, 2023). The camera was deployed on 13 Oct. 2022, adjacent to the weather station and eddy covariance system on a tripod, 2.16 m above the ground, facing towards the south-southwest to minimize self-shadow. The field is dryland no-till and was planted with winter pea on 30 Aug. 2022, following winter wheat. Over the Fall/Winter, the camera primary view soil, residue and snow. Emergence occurred in the last week of March. In the camera footprint, there were peas and volunteer wheat, which began to senesce in mid-June. The camera was removed prior to harvest at the end of July.

### Conservation and Production Research Laboratory, Bushland, Texas

The second site where an additional DSC was deployed was the Conservation and Production Research Laboratory (CPRL), located in Bushland, Texas (35° 11′ N, 102° 6′ W, 1170 m above mean sea level). The location is semiarid featuring strong regional advection, a mean annual temperature of 13.4° C., precipitation of 489 mm (NOAA Climate Data Online, 2023). The soil is a Pullman clay loam (fine, mixed, superactive, thermic Torrertic Paleustoll (NRCS Web Soil Survey, 2023). The field location was adjacent to a large weighing monolithic lysimeter. Four lysimeters were located in the centers of ˜4.4-ha fields that were approximately square, and the fields were arranged in a square pattern. The lysimeters and fields were designated northeast (NE), northwest (NW), southeast (SE) and southwest (SW). The DSC was located at the NE lysimeter, mounted on a horizontal aluminum pole (25 mm nominal diameter) and viewed the center of a crop row at nadir at 1.83 m above the soil surface. The DSC initially viewed bare soil with sparse corn residue left from the previous season. The field was planted in cotton on 9 May 2023 in east-west oriented rows spaced 0.76 m. The field was irrigated by subsurface drip, where drip laterals were spaced 1.52 m and buried 0.30-m deep below the soil surface in alternate interrows. The nearest drip lateral under the soil surface within in the DSC view was 0.26 m north of the crop row center.

### Device Design and Calibration

The DSC was comprised of four key components: a color camera (Sony IMX477); a thermal camera (FLIR Lepton 3.5); a computer system (Raspberry Pi Model 4); and a power supply. The control, computation, and communication were performed by a Raspberry Pi Model 4 B with 8 GB of RAM (Raspberry Pi Foundation, 2023). Power supplied to the Raspberry Pi was by a 5V, 3A DIN rail mounted AC-DC converter, and the Raspberry Pi was outfitted with a fan-equipped heatsink. Color images were taken with the Raspberry Pi HQ camera (Raspberry Pi High Quality Camera, 2023), as well as a Sony IMX477 with 12 megapixel (MP) maximum resolution and a lens providing 630 field of view (Adafruit, 2023). Initially, the DSC was set to collect images with resolution of 1280×960 every half hour (referred to as V1), but this was later updated to images of resolution 1920×1440, collected hourly (referred to as V2) to better detect finer-textured residue and germinating vegetation. The thermal camera was a FUR Lepton 3.5, a 160×120 pixel radiometric camera with a 57° field of view (Teledyne, 2023). Both cameras and the Pi were mounted on a 3D printed custom DIN rail adapter. All components were housed in a polycarbonate enclosure with a DIN-rail for mounting and a 100-mm thermal window (downward facing), permitting a clear view in both color and thermal bands. Camera control software was written in Python, and this was used primarily, except for a period when a C++version was tested at CAF to solve some data corruption issues described later in this disclosure.

Three calibration methods were employed to translate the thermal camera readings into physical temperatures, two in the laboratory and one in the field. This was important due to the influence of the thermal window not accounted for in the default calibration. At CAF, a reference black body capable of displaying its temperature (Wahl, 2023) was employed in an air-conditioned lab, kept at 22° C. It was initially chilled to 10° C., then allowed to warm while being monitored by the DSC, acquiring images every minute from a distance of approximately 20 cm. Once at room temperature, it was heated to 40° C. using a hot air gun, then allowed to cool while being monitored. After data collection, the temperatures were taken from the color images, and compared with the mean readings over the black body target from the thermal images, and a linear regression was fit (Table 1).

At CPRL, the DSC was mounted on a vertical pole over a water bath. The distance between the lens and the surface of the water bath was approximately 55 cm. Readings were taken over a 72 hour period while the target bath water temperature ranged from 22.3 to 59.9° C. The temperature readings were from a type-T thermocouple immersed in the water bath and connected to a datalogger (model CR300, Campbell Scientific, Logan, UT), which sampled measurements every 5 s and averaged and stored temperature data every 1 minute. Temperature readings from the thermocouple and thermal camera were paired based on the time stamps from the datalogger and the DSC device, respectively. As at CAF, linear regression was used to produce the calibration equation (Table 1).

Finally, a calibration was conducted at CAF to account for the effect of changing enclosure temperature. This used the black body, placed approximately 30 cm from the DSC under a 100 W heat lamp, and allowed to take images as both heated up to 38.5° C., capturing images on 5-minute intervals. Then, both the DSC and blackbody were placed into a walk-in cooler and allowed to cool to 7.3° C. Additionally, two measurements were taken outdoors with the blackbody under the tower-mounted DSC, but these were excluded from the calibration since the calibration target is smaller than 10×10 pixels, FLIR's minimum recommendation. The resultant data was processed in the same fashion as the CAF indoor calibration, but fit with a piecewise linear curve. This curve was applied on both camera versions (V1 and V2) as it most accurately accounted for environmental effects.

TABLE 1
Calibration linear regression translating raw FLIR
values to temperature in K using different methods.

| Calibration | |
|---|---|
| Method | Equation(s) |
| CAF | 0.0282x − 536.3049 |
| CPRL | 0.0251x − 451.0125 |
| Enclosure | 0.0097x + 7.8290 if x < 29655.1850 |
| effect | 0.03140x − 636.3688 if 29655.1851 ≤ x < 29811.2450 |
| | 0.0092x + 26.3804 if x ≥ 29811.2450 |

### Image Processing

Color images were manually labeled to serve as training data for the machine vision algorithm for segmentation. Labeling was performed using open software, ImageJ (Rasband, 2023) by first creating an image with soil, residue, vegetation, and snow labels, then a separate image with sunlit and shadow labels. The two labeled images were combined to create an image with the eight possible classes. Images were selected throughout the season to ensure a good diversity of vegetation cover and lighting conditions. Ultimately 44 images were selected from the CAF V1 camera, 10 from the CPRL V1, 34 from the CAF V2, and 6 from the CPRL V2. Data training sets from both locations were combined, but the V1 and V2 were treated separately, due to the difference in resolution. This resulted in two separate datasets, V1 and V2, with different resolutions.

The model used for image segmentation was a multi-layer perceptron, a form of neural network (Murtagh, 1991) and the process for training and testing corresponds to that shown in FIG. 3. All image processing was conducted using SciNet, the USDA-ARS computing cluster. An initial set of features (individual pixel values, Gaussian blur at two scales, standard deviation at three scales, linear binary pattern at three scales, and Laplacian at three scales) were calculated for each image in the training data in each of nine bands (red, green, blue, hue, saturation, value, and L*a*b* colorspace components). OpenCV was used to calculate features and Scikit-learn was used for the segmentation model training and testing (The OpenCV Reference Manual, 2023; Pedregosa et al., 2011). These 108 features ((9 bands×3 textures×3 scales)+(9 bands×1 pixel value)+(9 bands×blur at 2 scales)) for each pixel, and corresponding pixel labels, were split into 80% for training and 20% for testing. The training set was first scaled. Next, principal component analysis was applied to reduce the feature set, with the components accounting for a certain percentage of the variance retained. The reduced feature set (69 for V1, 70 for V2) was fed into a multilayer perceptron (MLP) with three hidden layers. The hyperparameters of the MLP (nodes per layer and activation function) and the percentage used in the PCA cutoff, were optimized (Table 2) using a grid search. Finally, the optimized model was evaluated with the test set, using test statistics of weighted F1 and accuracy scores (Table 3).

TABLE 2
MLP hyperparameters after optimization.

| | Nodes | Nodes | Nodes | | PCA |
|---|---|---|---|---|---|
| Version | layer 1 | layer 2 | layer 3 | Activation | Cutoff |
| V1 | 432 | 117 | 32 | logistic | 0.999 |
| V2 | 864 | 166 | 32 | logistic | 0.999 |

TABLE 3
Model performance metrics on test set.

| Camera Version | F1 | Accuracy |
|---|---|---|
| V1 | 0.8922 | 0.8923 |
| V2 | 0.8840 | 0.8846 |

When processing the thermal and color images, after the trained model was used to label the color image, the thermal image was registered to match up with the color image. To accomplish this, we placed a foil-covered PVC quadrat, 60 cm by 60 cm, underneath the camera in the field. The foil provides a low emissivity so it is distinct in both the thermal and color images. The pixel locations of the corners of the reference were measured in both images in a three-step process, all conducted in post-processing on SciNet. First, both color and thermal images were processed using adaptive thresholding to extract the square. Then, the Hough line fitting method (Duda & Hart, 1972) was used to detect lines passing through the sides of the quadrat. Finally, the intersections of the lines provided the corners' coordinates. The list of corners in the color and thermal images were used to calculate the affine transform to warp the thermal image to match the color image. Additionally, the reference was masked from the images because it did not provide useful data. At CAF, this reference remained in the field from installation to the point when it was obscured by vegetation (26 May 2023), while at CPRL, it was placed only for three hours on 23 Jun. 2023. It was necessary for such a long period at CAF due to frequent high winds that occasionally moved the camera position.

The thermal image when registered with the labelled color image provided the temperature of the eight different classes. The image labels acted as masks on the thermal image to acquire average temperatures of each component of interest (sunlight vegetation, sunlit residue, sunlit soil, sunlit snow, shaded vegetation, shaded residue, shaded soil, and shaded snow, etc.). The component temperatures were compiled over the season to create a time series. At night, the thermal images were useful to collect component temperature data, however, the color images were not, therefore the labels determined near the previous day's solar noon were used to classify components of interest. The difference being at night, all sunlit portions were reclassified as shadow.

### Calculating E, T, and ET

The original TSEB formulation was based on initializing canopy latent heat flux using the Priestley-Taylor equation (TSEB-PT), followed by solving the energy balance iteratively:

$$\text{?} \qquad ( 7 )$$

$$\text{?} \text{indicates text missing or illegible when filed}$$

where LE_{CI} is the initial canopy latent heat flux (W m⁻²), R_{N,C} is the net radiation to the canopy (W m⁻²), α_{PT} is the Priestley-Taylor parameter (α_{PT}=−1.26), f_{G} is the fraction of green vegetation (f_{G}=1.0 before senescence), A is the slope of the saturation vapor pressure-temperature relation (kPa ° C.⁻¹), γ is the psychrometric constant (kPa ° C.⁻¹), and the sign convention is positive toward the canopy. The TSEB-PT was tested at CPRL where soil evaporation (E), plant transpiration (T), and total evapotranspiration (ET) were measured by microlysimeters, sap flow gauges, and large weighing lysimeters, respectively, for irrigated cotton. The microlysimeters and sap flow gauges were located ˜30 m from the large weighing lysimeters in a ˜10-ha field, and E, T, and ET calculated by the TSEB-PT were compared to field measurements. The TSEB-PT consistently overestimated and underestimated E and T, respectively, compared with measurements, with root mean square errors up to 4 mm d⁻¹. Discrepancies between calculated and measured ET had a root mean square error of 3.2 mm d⁻¹. The respective over- and underestimates of E and T resulted because in the TSEB-PT, vapor pressure deficit was assumed constant. However, vapor pressure deficit in the semiarid climate of CPRL may vary by up to 5 kPa diurnally.

To mitigate the large discrepancies in E and T partitioning, scientists and engineers at CPRL reformulated the TSEB where initial canopy latent heat flux was calculated by the Penman-Monteith equation (TSEB-PM), followed by a similar iterative procedure to solve the energy balance:

$$\text{?} \qquad ( 8 )$$

$$\text{?} \text{indicates text missing or illegible when filed}$$

where ρ is the air density (kg m⁻³), C_{P} is the specific heat of air (assumed constant at 1013 J kg⁻¹ K⁻¹), γ*=γ(1+r_{C}/r_{A}), r_{C} is the bulk canopy resistance (s m⁻¹), r_{A} is the aerodynamic resistance between the canopy and the air above the canopy (s m⁻¹), e_{S} and e_{A} are the saturation and actual vapor pressures of the air, respectively (i.e., the vapor pressure deficit; kPa), and all other terms are as defined previously. The Penman-Monteith equation was used because vapor pressure deficit is included in the aerodynamic term, therefore accounting for its diurnal variation. Using the new TSEB-PM version, root mean square errors were reduced to 0.54 and 0.87 mm d⁻¹ for calculated vs. measured E and T, respectively, and 0.63 mm d⁻¹ for calculated vs. measured ET. From these results, it was concluded that the TSEB-PM version developed at CPRL was an improvement over the original TSEB-PT version. Therefore, the TSEB-PM version has been used at CPRL and CAF.

### Results and Discussion

### Field Test Results

Field deployment of the cameras highlighted some benefits and problems with the DSC. Firstly, the thermal camera frequently produced corrupted images, as the serial peripheral interface (SPI) protocol is sensitive to disruptions. In an enclosure outdoors, with vibration and electronic noise nearby, the noise level is high. C++ code was implemented using interrupts to maintain effective communication, but severe disruptions in the SPI resulted in lost images, and the problem was only recoverable by completely powering down the Lepton. Another field problem was difficulty in removing the camera for maintenance—this was solved with external connectors for data and power. Focusing the color camera manually was tedious and time-consuming in the field, and accessing it disturbed the crop in the field of view. Finally, severe wind at the CAF location (>100 kph) moved the crossarm from South-Southwest to East. Guy wires solved this issue.

### Segmentation

The hyperparameters of the trained models (V1 and V2) are given in Table 2 and the weighted F1 and accuracy scores in Table 3. Performance was similar at both resolutions (1280×960 for V1, 1920×1440 for V2), with weighted F1 and accuracy scores around 0.89 for both, but higher resolution was easier to label for training data. The most frequent mistakes in V1 were confusing soil for residue and vice-versa, and confusing sunlit and shaded soil. In V2, again, soil and residue were confused, as well as sunlit and shaded vegetation. In both cases, the confused classes were very similar visually. A possible solution may be to incorporate predictions from previous times, along with solar angles—a pixel is more likely to be soil if it was soil in the previous image, for example. The confusion between soil and residue was more evident in the CPRL images, where some of the soil surface exhibited textural features close to residue. In the CAF images, flowers were incorrectly identified as snow. As expected, in the both the CAF images and the CPRL images, vegetation is significantly cooler than soil and residue.

### Temperature

The values generally followed the correct diurnal cycles, with sunlit fractions peaking and shaded portions bottoming out at solar noon. They also correctly follow the trend of increasing vegetation fraction as the season progressed. Temperatures were cooler for the vegetation portion and shadowed portions. Residue tended to be hotter than bare soil since dry residue cannot evaporatively cool. The bare soil temperature measured by IR thermometer at CPRL matches the temperatures from the DSC, except it is higher during the day, where dry soil is hotter than an irrigated, vegetated surface.

### CONCLUSIONS

We have developed and tested a combined thermal and color imaging system called a dual smart camera (DSC). The DSC can measure separate temperatures of soil, residue, vegetation, and snow, in both shadowed and sunlit fractions. The DSC was field tested for the full growing season of winter peas in Eastern Washington and the spring and summer growing season of cotton in the Panhandle of Texas. The classification accuracy of the image segmentation to identify the different fractions of sunlit and shaded vegetation, soil, residue, and snow was near 0.89 at two different imaging resolutions, and the component temperatures followed correct diurnal and seasonal patterns. Future work could improve the accuracy by using semantic segmentation, reducing communication errors with the thermal imager, and relating temperatures to absolute measures of crop stress, like the ET and CWSI. We will continue to test and improve the DSC in different crops at the two sites.

Field cropping systems face increasingly severe variations in atmospheric demand and precipitation. These challenges require either breeding drought-tolerant cultivars, prescription irrigation, or identifying other sources of stress which are hard to distinguish from heat stress. Accordingly, understanding heat and water stress on field crops is an increasingly important consideration in management decisions. In dryland cropping systems, knowledge of heat stress can help detect drought tolerant cultivars and disambiguate other sources of stress, such as nutrient deficiencies, as well as allow growers to better assess planting and harvest dates to maximize use of stored water. Meanwhile, in irrigated crops, precision irrigation through stress monitoring has proven an effective approach to save water resources. For example, detection of crop water stress can be used to regulate irrigation scheduling in dry regions. In either case, surface temperature measurements can act as an indicator of crop water stress, however, current crop water stress monitoring and evapotranspiration (ET) estimation typically relies on approximations based on single radiometer measurements rather than imagery, but methods like infrared thermometry give a mix of crop and soil temperatures that may be sunlit or shaded. For example, thermal radiometers measuring a single value over a fixed footprint capture a mix of soil and vegetation temperatures, limiting usefulness during the early season with low vegetation cover.

Thermal imaging offers a straightforward solution. Several recent studies have explored new, inexpensive thermal cameras in vineyards (Garcia-Tejero et al., 2016; Zhou et al., 2022) and almonds (Gimenez-Gallego et al., 2021) but viewed the plants in profile, so were incompatible with grain crops, and did not show long field deployments. In accordance with some embodiments of the present invention, on the other hand, a downward-looking image is captured by the DSC, so is compatible with grain crops, and long field deployments enable a time series of color and thermal images to capture the target of interest over time.

Combined thermal and visible imagery allows for more accurate ET assessment in early growth stages, as well as identification of crop biotic stressors (e.g., disease or weeds). Such imagers are known in the prior art but are typically expensive and, with rare exception, are not adapted for agricultural applications. Two such exceptions, as noted above, are Osroosh et al. (2018) and Drew et al. (2019). Osroosh et al. developed a low-cost thermal-RGB imager for use in agricultural crop monitoring applications. In Osroosh et al., the image-processing algorithm outputs the average temperature of sunlit leaves and percent canopy coverage. Drew et al. developed a multi-band sensor for crop temperature measurement that combines a miniature long wavelength infrared (LWIR) camera with a visible, or red-green-blue (RGB), camera. In Drew et al., a processing algorithm outputs a temperature measurement more representative of the crop canopy by removing shaded areas and soil from thermal images. Applicants are unaware of any combined thermal and visible imagers known in the prior art that include the necessary image processing algorithms for crop water stress monitoring and ET estimation. In accordance with some embodiments of the present invention, on the other hand, provides that necessary image processing algorithm.

In digital image processing and analysis known in the prior art, image segmentation classifies each pixel by first extracting color and texture features, and then an algorithm is applied to properly label a given pixel. Some classification methods include random forests (Feng, 2015), support vector machines (Guerrero et al., 2012), thresholding (Wang et al., 2013), and semantic segmentation (Sodjinou et al., 2022). Applicants are unaware of any digital image processing and analysis known in the prior art that include the necessary feature extraction and image segmentation for crop water stress monitoring and ET estimation. In accordance with some embodiments of the present invention, on the other hand, provides that necessary feature extraction and image segmentation.

In prior art systems, extracting vegetation temperature is more difficult in a complex image containing soil, vegetation, residue, and snow, in both sun and shade. The difficultly is in properly labelling each pixel over changing lighting and plant growth stages. This difficulty is overcome, in accordance with some embodiments of the present invention, by segmenting a color image into surface temperature components based on features extracted from the color image, wherein the surface temperature components are selected from the group consisting of sunlit soil, sunlit residue, sunlit vegetation, sunlit snow, shaded soil, shaded residue, shaded vegetation, and shaded snow.

The content of certain images (e.g., a downward-looking image of a no-till field crop) can be challenging to segment, due to the fine features, and similarities between the appearance of soil, residue, and senescing vegetation. Images such as these are successfully segmented, in accordance with some embodiments of the present invention, by first extracting numerous color and texture features. In accordance with some embodiments, for each pixel, features extracted include an individual pixel value, Gaussian blur at two scales, standard deviation at three scales, linear binary pattern at three scales, and Laplacian at three scales in each of nine bands, wherein the nine bands include red, green, blue, hue, saturation, value, and L*a*b* colorspace components. Hence, for each pixel, 108 features ((9 bands×3 textures×3 scales)+(9 bands×1 pixel value)+(9 bands×blur at 2 scales)) are obtained.

While this invention may be embodied in many different forms, there are described in detail herein specific preferred embodiments of the invention. The present disclosure is an exemplification of the principles of the invention and is not intended to limit the invention to the particular embodiments illustrated. All patents, patent applications, scientific papers, and any other referenced materials mentioned herein are incorporated by reference in their entirety. Furthermore, the invention encompasses any possible combination of some or all of the various embodiments and characteristics described herein and/or incorporated herein. In addition, the invention encompasses any possible combination that also specifically excludes any one or some of the various embodiments and characteristics described herein and/or incorporated herein.

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein). The invention illustratively disclosed herein suitably may be practiced in the absence of any element (e.g., method (or process) steps or composition components) which is not specifically disclosed herein. Thus, the specification includes disclosure by silence (“Negative Limitations In Patent Claims,” AIPLA Quarterly Journal, Tom Brody, 41(1): 46-47 (2013): “ . . . . Written support for a negative limitation may also be argued through the absence of the excluded element in the specification, known as disclosure by silence . . . . Silence in the specification may be used to establish written description support for a negative limitation. As an example, in Ex parte Lin [No. 2009-0486, at 2, 6 (B.P.A.I. May 7, 2009)] the negative limitation was added by amendment . . . . In other words, the inventor argued an example that passively complied with the requirements of the negative limitation . . . was sufficient to provide support . . . . This case shows that written description support for a negative limitation can be found by one or more disclosures of an embodiment that obeys what is required by the negative limitation . . . .”

Unless otherwise indicated, all numbers expressing quantities of ingredients, properties such as molecular weight, reaction conditions (e.g., reaction time, temperature), percentages and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. As used herein, the term “about” refers to a quantity, level, value, or amount that varies by as much as 10% to a reference quantity, level, value, or amount. For example, about 1.0 g means 0.9 g to 1.1 g and all values within that range, whether specifically stated or not.

### REFERENCES

Adafruit. (2023). 6 mm 3MP Wide Angle Lens. Retrieved from https://www.adafruit.com/product/4563
Agam, N., Kustas, W. P., Evett, S. R., Colaizzi, P. D., Cosh, M., & McKee, L. G. (2012). Soil heat flux variability influenced by row direction in irrigated cotton. Adv. Water Resour., 50, 31-40. http://dx.doi.org/10.1016/j.advwatres.2012.07.017
Campbell, G. S., & Norman, J. M. (1998). An Introduction to Environmental Biophysics (2nd ed.). New York, N.Y.: Springer-Verlag. http://dx.doi.org/10.1007/978-1-4612-1626-1
Colaizzi, P. D., O'Shaughnessy, S. A., Gowda, P. H., Evett, S. R., Howell, T. A., Kustas, W. P., & Anderson, M. C. (2010). Radiometer footprint model to estimate sunlit and shaded components for row crops. Agron. J., 102(3), 942-955. http://dx.doi.org/10.2134/agronj2009.0393
Colaizzi, P. D., Kustas, W. P., Anderson, M. C., Agam, N., Tolk, J. A., Evett, S. R., Howell, T. A., Gowda, P. H., & O'Shaughnessy, S. A. (2012a). Two-source energy balance model estimates of evapotranspiration using component and composite surface temperatures. Adv. Water Resour., 50, 134-151. http://dx.doi.org/10.1016/j.advwatres.2012.06.004.
Colaizzi, P. D., Evett, S. R., Howell, T. A., Li, F., Kustas, W. P., & Anderson, M. C. (2012b). Radiation model for row crops: I. Geometric model description and parameter optimization. Agron. J., 104(2), 225-240. http://dx.doi.org/10.2134/agronj2011.0082
Colaizzi, P. D., N. Agam, J. A. Tolk, S. R. Evett, T. A. Howell, Sr., S. A. O'Shaughnessy, P. H. Gowda, W. P. Kustas, and M. C. Anderson. (2016a). Advances in the two-source energy balance model: Partitioning of evaporation and transpiration for row crops. Trans. ASABE 59(1): 181-197. DOI 10.13031/trans.59.11215
Colaizzi, P. D., Evett, S. R., Agam, N., Schwartz, R. C., & Kustas, W. P. (2016b). Soil heat flux calculation for sunlit and shaded surfaces under row crops: 1. Model development and sensitivity analysis. Agric. Forest Meteorol., 216, 115-128. http://dx.doi.org/10.1016/j.agrformet.2015.10.010
Colaizzi, P. D., S. A. O'Shaughnessy, S. R. Evett, and R. B. Mounce. (2017). Crop evapotranspiration calculation using infrared thermometers aboard center pivots. Agric. Water Manage. 187: 173-189. http://dx.doi.org/10.1016/j.agwat.2017.03.016
Drew, P. L., Sudduth, K. A., Sadler, E. J., Thompson, A. L. (2019). Development of a multi-band sensor for crop temperature measurement. Computers and Electronics in Agriculture, 162, 269-280. doi:10.1016/j.compag.2019.04.007
Duda, R. O., & Hart, P. E. (1972). Use of the Hough transformation to detect lines and curves in pictures. Communications of the ACM, 15(1), 11-15. doi:10.1145/361237.361242
Feng, Q. L. (2015). UAV remote sensing for urban vegetation mapping using random forest and texture analysis. Remote sensing, 7(1), 1074-1094. doi:10.3390/rs70101074
Garcia-Tejero, I. F., Costa, J. M., Egipto, R., Durán-Zuazo, V. H., Lima, R. S., Lopes, C. M., & Chaves, M. M. (2016). Thermal data to monitor crop-water status in irrigated Mediterranean viticulture. Agricultural Water Management, 176, 80-90. doi:10.1016/j.agwat.2016.05.008
Gimenez-Gallego, J., González-Teruel, J. D., Soto-Valles, F., & Jiménez-Buendia, M. (2021). Intelligent thermal image-based sensor for affordable measurement of crop canopy temperature. Computers and Electronics in Agriculture, 188. doi:10.1016/j.compag.2021.106319
Guerrero, J. M., Pajares, G., Montalvo, M., Romeo, J., & Guijarro, M. (2012). Support vector machines for crop/weeds identification in maize fields. Expert Systems with Applications, 39(2), 11149-11155. doi:10.1016/j.eswa.2012.03.040
Huggins, D. R. (2015). The Cook Agronomy Farm LTAR: Knowledge Intensive Precision Agro-ecology. AGU Fall Meeting Abstracts. San Francisco, CA.
Kleinman, P., Spiegal, S., Rigby, J., Goslee, S., Baker, J., Bestelmeyer, B., . . . Duncan, E. (2018). Advancing the sustainability of US agriculture through long-term research. Journal of Environmental Quality, 47(6), 1412-1425. doi:10.2134/jeq2018.05.0171
Kool, D., Ben-Gal, A., Agam, N., çimnnek, J., Heitman, J. L., Sauer, T. J., & Lazarovitch, N. (2014). Spatial and diurnal below canopy evaporation in a desert vineyard: Measurements and modeling. Water Resour. Res., 50(8), 7035-7049. http://dx.doi.org/10.1002/2014WR015409
Kustas, W. P., & Norman, J. M. (1999). Evaluation of soil and vegetation heat flux predictions using a simple two-source model with radiometric temperatures for partial canopy cover. Agric. Forest Meteorol., 94(1), 13-29. http://dx.doi.org/10.1016/S0168-1923(99)00005-2
Murtagh, F. (1991). Multilayer perceptrons for classification and regression. Neurocomputing, 2(5-6), 183-197. doi:10.1016/0925-2312(91)90023-5 NOAA Climate Data Online. (2023). Retrieved from https://www.ncei.noaa.gov/access/us-climate-normals
Norman, J. M., Kustas, W. P., & Humes, K. S. (1995). Source approach for estimating soil and vegetation energy fluxes in observations of directional radiometric surface temperature. Agric. Forest Meteorol., 77(3-4), 263-293. http://dx.doi.org/10.1016/0168-1923(95)02265-Y NRCS Web Soil Survey. (2023). Retrieved from https://websoilsurvey.nres.usda.gov/app/
Osroosh, Y., Khot, L. R., and Peters, R. T., (2018). Economical thermal-RGB imaging system for monitoring agricultural crops. Computers and Electronics in Agriculture, 147, 34-43. doi:10.1016/j.compag.2018.02.018
Pedregosa, F., Varoquaux, G., Gramfort, A., Michel, V., Thirion, B., Grisel, O., . . . Prettenhofer, P. (2011). Scikit-learn: Machine Learning in Python. Journal of Machine Learning Research, 12, 2825-2830.
Rasband, W. (2023). ImageJ, U. S. National Institutes of Health, Bethesda, Maryland, USA. Retrieved from https://imagej.nih.gov/ij/
Raspberry Pi Foundation. (2023, 7 2). Retrieved from https://www.raspberrypi.org/
Raspberry Pi High Quality Camera. (2023). Retrieved from https://www.raspberrypi.com/products/raspberry-pi-high-quality-camera/
Sherstinsky, A. (2020). Fundamentals of recurrent neural network (RNN) and long short-term memory (LSTM) network. Physica D: Nonlinear Phenomena, 404, 132306. https://doi.org/10.1016/j.physd.2019.132306
Sierra Wireless. (2023). AirLink Cellular Routers. Retrieved from https://www.sierrawireless.com/router-solutions
Sodjinou, S. G., Mohammadi, V., Mahama, A. T., & Gouton, P. (2022). A deep semantic segmentation-based algorithm to segment crops and weeds in agronomic color images. Information Processing in Agriculture, 9(3), 355-364. doi:10.1016/j.inpa.2021.08.003
Teledyne. (2023). LWIR Micro Thermal Camera Module: FLIR Lepton. Retrieved from https://www.flir.com/products/lepton/?vertical=microcam&segment=oem
The OpenCV Reference Manual. (2023, 7 2). Retrieved from https://docs.opencv.org/4.x/
Wahl. (2023). Ambient Portable Black Body Calibration Unit. Retrieved from https://www.wahlheatspy.com/product/hsicbb-p/
Wang, Y. W., Zhang, G., & Wang, J. (2013). Estimating nitrogen status of rice using the image segmentation of GR thresholding method. Field Crops Research, 33-39. doi:10.1016/j.fcr.2013.04.007
Zhou, Z., Diverres, G., Kang, C., Thapa, S., Karkee, M., Zhang, Q., & Keller, M. (2022). Ground-based thermal imaging for assessing crop water status in grapevines over a growing season. Agronomy, 12(2). doi:10.3390/agronomy12020322

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

What is claimed is:

1. A computer-implemented method of estimating evapotranspiration (ET) using thermal images and optical images, comprising:
acquiring a color image that captures a target of interest, wherein the target of interest is associated with an agricultural crop;
acquiring a thermal image that captures the target of interest at substantially the same time as the color image;
extracting one or more features from the color image, wherein extracting one or more features from the color image includes extracting color features and/or texture features;
segmenting the color image into surface temperature components based on the one or more features extracted from the color image, wherein the surface temperature components are selected from the group consisting of sunlit soil, sunlit residue, sunlit vegetation, sunlit snow, shaded soil, shaded residue, shaded vegetation, and shaded snow;
co-registering the color image and the thermal image to provide a registered thermal image;
assigning component temperatures by applying component masks to the registered thermal image to assign a component temperature to each of the surface temperature components;
estimating ET based on the component temperatures using an energy balance model or other ET model.

2. The method of claim 1, wherein the color image and the thermal image are acquired every about 0.5-1 h to provide a color image time series and a thermal image time series capturing the target of interest over time.

3. The method of claim 2, wherein assigning component temperatures includes constructing a time series TS-Component for each of the surface temperature components.

4. The method of claim 1, wherein extracting features includes extracting, for each pixel, features including an individual pixel value, Gaussian blur at two scales, standard deviation at three scales, linear binary pattern at three scales, and Laplacian at three scales in each of nine bands, wherein the nine bands include red, green, blue, hue, saturation, value, and L*a*b* colorspace components.

5. The method of claim 1, wherein segmenting the color image includes performing classification by inputting a reduced feature set into a multi-layer perceptron.

6. The method of claim 5, further comprising training and testing the multi-layer perceptron using images of a cropping system representative of the agricultural crop.

7. The method of claim 1, wherein co-registering the color image and the thermal image includes using a thermally reflective object that appears in the color image and the thermal image, matching a plurality of features of the thermally reflective object between the color image and the thermal image, and using the plurality of features of the thermally reflective object to calculate an affine transform to warp the thermal image to match the color image.

8. The method of claim 1, wherein the energy balance model is a two-source energy balance (TSEB) model.

9. The method of claim 1, wherein the energy balance model is an extension of a two-source energy balance (TSEB) model extended to include at least one additional source.

10. The method of claim 1, wherein the other ET model utilizes a recurrent neural network (RNN) that estimates ET based on the component temperatures and local meteorological time series.

11. A system for estimating evapotranspiration (ET) using thermal and optical images, comprising:
an optical camera for acquiring a color image that captures a target of interest, wherein the target of interest is associated with an agricultural crop;
a thermal camera for acquiring a thermal image that captures the target of interest at substantially the same time as the color image;
a computing device operatively connected to the optical camera and the thermal camera to receive the color image and the thermal image and to perform a method comprising:
extracting one or more features from the color image, wherein extracting one or more features from the color image includes extracting color features and/or texture features;
segmenting the color image into surface temperature components based on the one or more features extracted from the color image, wherein the surface temperature components are selected from the group consisting of sunlit soil, sunlit residue, sunlit vegetation, sunlit snow, shaded soil, shaded residue, shaded vegetation, and shaded snow;
co-registering the color image and the thermal image to provide a registered thermal image;
assigning component temperatures by applying component masks to the registered thermal image to assign a component temperature to each of the surface temperature components;
estimating ET based on the component temperatures using an energy balance model or other ET model.

12. The system of claim 11, wherein the color image and the thermal image are acquired every abut 0.5-1 h to provide a color image time series and a thermal image time series capturing the target of interest over time.

13. The system of claim 12, wherein assigning component temperatures includes constructing a time series TS-Component for each of the surface temperature components.

14. The system of claim 11, wherein extracting features includes extracting, for each pixel, features including an individual pixel value, Gaussian blur at two scales, standard deviation at three scales, linear binary pattern at three scales, and Laplacian at three scales in each of nine bands, wherein the nine bands include red, green, blue, hue, saturation, value, and L*a*b* colorspace components.

15. The system of claim 11, wherein segmenting the color image includes performing classification by inputting a reduced feature set into a multi-layer perceptron trained on images of a cropping system representative of the agricultural crop.

16. The system of claim 11, wherein co-registering the color image and the thermal image includes using a thermally reflective object that appears in the color image and the thermal image, matching a plurality of features of the thermally reflective object between the color image and the thermal image, and using the plurality of features of the thermally reflective object to calculate an affine transform to warp the thermal image to match the color image.

17. The system of claim 11, wherein the optical camera, the thermal camera, and the computing device are packaged together in a single housing.

18. The system of claim 11, wherein the energy balance model is a two-source energy balance (TSEB) model.

19. The system of claim 11, wherein the energy balance model is an extension of a two-source energy balance (TSEB) model that is extended to include at least one additional source.

20. A computer program product for estimating evapotranspiration (ET) using thermal images and optical images, the computer program product comprising a computer readable storage medium having program code embodied therewith, the program code executable by one or more processors, to perform a method comprising:
acquiring a color image that captures a target of interest, wherein the target of interest is associated with an agricultural crop;
acquiring a thermal image that captures the target of interest at substantially the same time as the color image;
extracting one or more features from the color image, wherein extracting one or more features from the color image includes extracting color features and/or texture features;
segmenting the color image into surface temperature components based on the one or more features extracted from the color image, wherein the surface temperature components are selected from the group consisting of sunlit soil, sunlit residue, sunlit vegetation, sunlit snow, shaded soil, shaded residue, shaded vegetation, and shaded snow;
co-registering the color image and the thermal image to provide a registered thermal image;
assigning component temperatures by applying component masks to the registered thermal image to assign a component temperature to each of the surface temperature components;
estimating ET based on the component temperatures using a two-source energy balance (TSEB) model or other ET model.
